(19) United States
(12) Patent Application Publication
(10) Pub. No.: US 2011/0104300 A1
(43) Pub. Date: May 5, 2011

(54) Methods for Screening Chemical, Biological, and Physical Agents for Disinfecting Infectious Norovirus

(75) Inventors: Myung L. Kim, Champaign, IL (US); David J. Schlesinger, Marshfield, WI (US)

(73) Assignee: KIM LABORATORIES, INC., Rantoul, IL (US)

(21) Appl. No.: 12/892,505

(22) Filed: Sep. 28, 2010

Related U.S. Application Data
(60) Provisional application No. 61/246,363, filed on Sep. 28, 2009.

(51) Int. Cl. C12Q 1/70; C40B 30/06; A01N 33/12; A01N 59/00; A01N 37/02; A01N 59/16; A01N 59/08; A01N 31/08; A01P 1/00

(52) U.S. Cl. 424/616; 435/5; 506/10; 514/642; 514/557; 424/618; 424/661; 424/680; 514/736

(57) ABSTRACT

The invention provides methods and kits for screening a test agent for antiviral activity against Norovirus. The methods are effective in terms of determining the amount of intact Norovirus while decreasing the amount of interfering RNA and PCR inhibitors within the test sample.

### PRIORITY

This application claims the benefit of U.S. Provisional application Ser. No. 61/246,363, filed Sep. 28, 2009, which is incorporated by reference herein in its entirety.

### BACKGROUND OF THE INVENTION

Norovirus is a major pathogen that is responsible for over 50% of all cases of gastroenteritis worldwide. According to the CDC, 23 million Americans contract Norovirus each year and in 2007 it was the leading confirmed cause of foodborne illness. This current threat to food safety has no intervention, vaccine, detection, or diagnostic method available. Transmission of Norovirus occurs through the fecal-oral route and can cause infection through person to person contact, which accounts for the majority of Norovirus outbreaks. To protect individuals from exposure to Norovirus and subsequent illness, disinfection is the only key preventive measure available thus far. For instance, disinfecting surface areas that come into contact with infected individuals could potentially decrease transmission of the disease. Although there are many sanitizing and disinfecting products that claim efficacy against Norovirus, none of these products actually have been tested for activity against Norovirus.

Since Norovirus cannot be grown in the laboratory and Norovirus positive stool samples (the only known source for infectious Norovirus) are difficult to obtain, researchers use surrogate viruses to test the disinfecting effects of chemical compounds. For example, Feline Calicivirus or Murine Norovirus can be easily detected or assayed using available animal tissue culture infection models or animal hosts. As such, most of the current agents that claim efficacy against Norovirus are tested against one of these surrogates using an infection tissue culture model. Since there is no method of testing for presence of Norovirus, the Environment Protection Agency (EPA) allows claims on Norovirus disinfecting efficacy if the agent has been shown effective against a surrogate virus. It is questionable, however, whether this efficacy can be applied to Norovirus. See e.g., Poschetto et al., Comparison of the sensitivities of noroviruses and feline calicivirus to chemical disinfection under field-like conditions. Appl. Environ. Microbiol. 73(17):5494-500, 2007; Doultree et al., Inactivation of feline calicivirus, a Norwalk virus surrogate. J. Hosp. Infect. 41(1):51-7, 1999; Belliot et al., Use of murine norovirus as a surrogate to evaluate resistance of human norovirus to disinfectants. Appl. Environ. Microbiol. 74(10):3315-8, 2008.

There remains a desire for methods for testing and screening chemical, biological, and physical agents for antiviral activity against infectious Norovirus.

### BRIEF SUMMARY OF THE INVENTION

In one embodiment, the invention provides a method of screening a test agent for antiviral activity against Norovirus. The method comprises contacting a Norovirus-positive sample with a test agent to form a Norovirus-test agent sample; adding neutralization buffer to the Norovirus-test agent sample to form a neutralization buffer sample; contacting the neutralization buffer sample with a solid support, and separating the solid support from the neutralization buffer sample. The amount of Norovirus associated with the solid support is determined, wherein a decrease in the amount of Norovirus in comparison to a control is indicative of antiviral activity of the test compound. The method can further comprise selecting a test agent that has at least a 1, 2, or 3 log reduction in the amount of Norovirus. The method can additionally further comprise contacting the neutralization buffer sample with one or more RNases at any time prior to contacting the neutralization buffer with the solid support (i.e., before and/or after addition of the neutralization buffer or before and/or after adding the test agent) or after contact with the solid support. The solid support can comprise a material that non-specifically binds viruses, such as polystyrene or polypropylene or a combination thereof. The solid support can comprise a directly or indirectly bound ligand specific for Norovirus such as a monoclonal antibody, polyclonal antibody, cell receptor, or a histo-blood group antigen (HBGA). The solid support can comprise immunomagnetic separation (IMS) beads, magnetic beads, hydrophobic beads, nitrocellulose, a filter, a plastic sheet or slide, glass beads, controlled pore silicate glass beads, a tube, a microtiter plate, a glass slide, a microchip array, a well or depression, ceramic, metal, resin, gel silicon, microspheres, or a combination thereof. The amount of Norovirus in the sample can be determined using microarray analysis, PCR amplification, RNA hybridization, gel electrophoresis, or combinations thereof. The amount of Norovirus in the sample can be determined using quantitative reverse transcription PCR (qRT-PCR). The Norovirus positive sample can be stool, vomitus, tissue, sputum, blood, plasma, serum, food, water, an environmental sample, a laboratory sample or any combinations thereof. The Norovirus positive sample can be from a human.

Another embodiment of the invention provides a kit for screening for a compound for antiviral activity against Norovirus comprising a solid support having a ligand that specifically binds Norovirus directly or indirectly bound to its surface, primers for amplification of Norovirus RNA, and a test or control sample comprising active, infectious Norovirus. The kit can comprise one or more RNases.

Even another embodiment of the invention provides method of screening a test agent for antiviral activity against Norovirus. The method comprises contacting a Norovirus-positive sample with a test agent to form a Norovirus-test agent sample; adding neutralization buffer to the Norovirus-test agent sample to form a neutralization buffer sample; and contacting the neutralization buffer sample with one or more RNases either (i) before the neutralization buffer is added; (ii) after the neutralization buffer is added; or (iii) before and after the neutralization buffer is added (including, e.g., addition of the one or more RNases before or after the test agent). The amount of Norovirus in the neutralization buffer sample is determined. The test agent has antiviral activity when the amount of Norovirus in the neutralization buffer sample is less than the amount of Norovirus in a control sample.

The method can further comprise selecting a test agent that has at least a 1, 2, or 3 or more log reduction in the amount of Norovirus. The amount of Norovirus in the sample can be determined using microarray analysis, PCR amplification, RNA hybridization, gel electrophoresis, quantitative reverse transcription PCR (qRT-PCR) or combinations thereof. The Norovirus positive sample can be stool, vomitus, tissue, sputum, blood, plasma, serum, food, water, an environmental sample, a laboratory sample or any combinations thereof. The Norovirus positive sample can be from a human.

Yet another embodiment of the invention provides a method of disinfecting a surface against Norovirus. The method comprises selecting a compound as a disinfectant for Norovirus by determining that the compound has antiviral activity against Norovirus according to the methods described herein and contacting the surface with the compound to disinfect the surface against Norovirus. The compound can be, e.g., ACTICIDE™ disinfectant.

### DETAILED DESCRIPTION OF THE INVENTION

### Noroviruses

The Norovirus used in the methods of the invention can be any type of Norovirus, for example, any strain, serotype or isolate of Norovirus that infects animals such as humans. The Norovirus can be of a genotype generally found in human hosts, such as genotypes GI, GII, and GIV, or any other genotype (e.g., GIII or GV). Particular strains of Norovirus that infect human hosts and mammalian hosts include, but are not limited to, Desert Shield virus (Hu/NLV/DSV395/1990/SR) (GenBank Accession number U04469), Lordsdale virus (Hu/NLV/LD/1993/UK) (GenBank Accession number X86557), Mexico virus (Hu/NLV/MX/1989/MX) (GenBank Accession number U22498), Norwalk virus (Hu/NLV/NV/1968/US) (GenBank Accession number M87661), Hawaii virus (Hu/NLV/HV/1971/US) (GenBank Accession number U07611), Snow Mountain virus (Hu/NLV/SMV/1976/US) (GenBank Accession number L23831), Southampton virus (Hu/NLV/SHV/1991/UK) (GenBank Accession number L07418), Alphatron (GenBank Accession number AF195847), murine NoV 1 (GenBank Accession number AY228235), porcine NoV SW918 (GenBank Accession number AB074893), bovine NoV Jena (GenBank Accession number AJ01109), bovine NoV Newbury Agent (GenBank Accession number AF097917), bovine Newbury Agent 1 strain (DQ013304), and bovine Nebraska strain (GenBank Accession number NC_{—}004064).

A Norovirus-positive sample can be any suitable sample. For example, the Norovirus-positive sample can be stool, vomitus, tissue, sputum, blood, plasma, serum, food, water, environmental sample or any combinations thereof. The sample can also be a sample prepared in the laboratory. A sample can be artificially prepared by injecting an isolated pure norovirus from its contaminated source into an artificial testing source (e.g., human or animal stool samples) to create a test sample. A sample can be obtained from any mammal, including, for example, humans, pigs, cattle, dogs, mink, mice, marine mammals, rabbits, and felines. In one embodiment of the invention, the Norovirus in the sample is present at about 10², 10³, 10⁴, 10⁵, 10⁶, 10⁷, 10⁸, 10⁹, 10¹⁰, 10¹¹, 10¹² or any range between about 10² and 10¹² (e.g., about 10⁶ to about 10⁹ or about 10⁷ to about 10⁹) virus particles per ml or mg of sample. The Norovirus-positive sample can be any suitable volume, for example, 0.1, 1, 5, 10, 15, 20, 50, 75, or 100 μl or μg, or 0.5, 1, 2, 5, 10, 50, 75, 100, 250, 500 ml or mg or more. Any range of sample between about 0.1 μl or μg and about 50 ml or mg can be used.

In methods of the invention a test agent is added to a positive Norovirus sample to form a Norovirus-test agent sample. A test agent is any compound, disinfectant, molecule, biological molecule (e.g., an antibody, protein, nucleic acid molecule, virion, microorganism), or physical change (e.g., change in temperature, pressure, oxygen concentration, other gas concentration, irradiation, pH) or combinations thereof. Following the addition of the test agent to the Norovirus positive sample, the Norovirus-test agent sample can optionally be incubated for a period of for about 1, 2, 3, 4, 5, 10, 15, 20, 25, 30, 35, 40, 45, 50, 55, 60, 75, 90, 105, or 120 minutes or any range of time between about 1 minute and about 120 minutes. The Norovirus-test agent sample can be incubated at any suitable temperature, for example, about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., or 37° C., or any range of temperatures between about 15° C. and 37° C.

### Neutralization Buffer

After the test agent is added to the Norovirus positive sample to form a Norovirus-test agent sample, a neutralization buffer (e.g., PBS or PBST) is added to form a neutralization buffer sample. The addition of the neutralization buffer halts the test agent's activity and allows for assessment of the antiviral activity of the test agent. The neutralization buffer can act by dilution of the test agent, which stops its disinfection action (e.g., PBS or PBST or other suitable buffer). Alternatively, a neutralization buffer can act by interacting with the test agent to stop its activity. In another embodiment, the intact Norovirus virions that survive the test agent can be separated from the test agent by separation using, e.g., a gel filtration column or other columnar separation. A neutralization buffer can act by dilution, interaction with the test agent, or both methods optionally in combination with columnar separation. In one embodiment of the invention the neutralization buffer is added at an excess volume of about 2, 10, 50, 100, 250, 300, 400, 500, 1,000, 2,000, 3,000, 4,000, 5,000, 10,000 or more times the volume or weight of the Norovirus-test agent sample. In one embodiment of the invention certain neutralization buffers known to interact with certain types of test agents and stop, inhibit or slow the disinfectant activity of the test agents can be used. Examples of such suggested neutralization buffers are provided in Table 1, although any other neutralization buffers or combinations of neutralization buffers can be used.

| Test Agent | Suggested Neutralization Buffer |
|---|---|
| Various quaternary ammonium | serum + 1% polysorbate 80 + 0.5% lecithin |
| compounds | |
| up to 0.5% of hydrogen peroxide | serum + 1% polysorbate 80 + 0.1% catalase + 0.01% |
| | sodium thiosulfate |
| 3.2% lactic acid | serum + 1% polysorbate 80 + 5% NaHCO3 + 5% |
| | HEPES |
| 0.176% orthophenylphenol, 0.045% | serum + 1% polysorbate 80 |
| para-tertiary amylphenol | |
| 21.41% potassium peroxymonosulfate, | serum + 1% polysorbate 80 |
| 1.5% sodium chloride | |
| 0.003% silver & 4.840% citric acid | serum + 1% polysorbate 80 + 5% NaHCO3 + 5% |
| | HEPES + 1 mm EDTA |
| 0.200% chlorine dioxide | serum + 1% polysorbate 80 + 0.01% sodium |
| | thiosulfate |

Optionally, the neutralization buffer sample is contacted with one or more RNases prior to or after contacting the neutralization buffer with a solid support.

### Solid Support and Ligands for Norovirus

The neutralization buffer sample is added to a solid support that comprises one or more ligands that specifically bind to Norovirus. The solid support can be any suitable solid support including, for example, immunomagnetic separation (IMS) beads, magnetic beads, hydrophobic beads, nitrocellulose, a filter, a plastic sheet or slide, glass beads, controlled pore silicate glass beads, a tube, a microtiter plate, a glass slide, a microchip array, a well or depression, ceramic, metal, resin, gel silicon, or microspheres.

One or more ligands that specifically bind Norovirus can be directly or indirectly bound or immobilized to the solid support. “Specific binding” or “specifically binds,” mean that a ligand exhibits appreciable affinity for Norovirus and, generally, does not exhibit significant cross-reactivity with other virions or organisms. A ligand that “does not exhibit significant cross-reactivity” is one that will not appreciably bind to an undesirable entity (e.g., a virus or organism other than Norovirus). Specific binding can be determined according to any art-recognized methodology, including, for example, Scatchard analysis and/or competitive binding assays. The ligand can be any suitable ligand that specifically binds Norovirus. For example, the ligand can be a monoclonal antibody, a polyclonal antibody, a cell receptor, a histo-blood group antigen (HBGA) or combinations thereof. Monoclonal antibodies specific for Norovirus include, for example, antibodies that are specific for SEQ ID NOs:1-16 of International Patent Application Publication WO 2008/005880, mAbN2C3 (Li et al., Virus Res. 151:142 (2010), and mAb MNV-S7 (Kitawaga et al., Exp. Anim. 59:47 (2010)). Polyclonal antibodies specific for Norovirus include, for example, antibodies specific for GI-1 and GII-4 Norovirus capsid proteins (Park et al, Appl. Environ. Microbiol. 74:4226 (2008)). HBGAs specific for Norovirus include, for example, A-type and H-type (Morton et al., Appl. Environ. Microbiol. 75:4641; Huhti et al., Arch. Virolog. (2010).

Alternatively, ligands that non-specifically bind any type of virus can be attached to or comprise the solid support (e.g., activated or non-activated polystyrene or polypropylene; Thermo IMMULON® microtiter plates or strips). “Non-specific binding” or “non-specifically binds,” mean that a ligand or solid support generally binds Norovirus and one or more other different types of viruses. Norovirus can then be distinguished from the other viruses at the quantification step where only Norovirus RNA is amplified. For example, beads, such as IMS beads or solid supports can be coated with polystyrene, activated polystyrene, polypropylene, activated polypropylene, or other encapsulation material and can bind Norovirus and one or more other viruses through non-specific protein-plastic interactions. Additionally, antibodies or other ligands that bind generally to viral capsid proteins or other viral proteins or components can be used as ligands to bind to Norovirus and one or more different viruses.

The neutralization buffer sample can be added to the solid support comprising the ligand and optionally incubated for a suitable period of time, for example, about 1, 2, 3, 4, 5, 10, 20, 30, 45, 60, 75, 90, 105, 120 minutes, 2.25, 2.5, 2.75, 3, 5, 10, or 24 hours or any range of time between about 1 minute and 24 hours). The neutralization buffer sample and solid support can be incubated at any suitable temperature, for example, at about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., or 37° C., or any range of temperatures between about 15° C. and about 37° C.

The use of a solid support in conjunction with a ligand specific for Norovirus, e.g., IMS beads conjugated to an antibody specific for Norovirus has several advantages. For example, the solid support can bind and isolate surviving and intact whole Norovirus from impurities such as free viral RNA (whether originated from direct action of the test agent or ubiquitously present in the test sample, e.g., infected human or animal stool). Additionally, the use of a solid support that can be washed or physically removed from the neutralization buffer sample allows for the removal of impurities such as PCR inhibitors, free contaminating RNA, and free RNA from broken Noroviruses. These impurities can cause inconsistent and unreliable results. Therefore, the method can distinguish between Norovirus RNA from Noroviruses that have been damaged by the test agent and Norovirus RNA from intact virions that survive contact with the test agent. Therefore, the use of the solid support provides a more accurate assessment of infective Norovirus at the stage of Norovirus quantification. Furthermore, the use of a solid support allows concentration of intact Norovirus from a large volume of test agent-neutralization buffer (e.g. about 1, 5, 10, 25, 40, 50, 75, 100, 250, 500, 750, 1,000 ml or any range between about 1 and about 1,000 ml) into a small quantity of volume (e.g., about 0.001, 0.01, 0.1, 0.5, 1.0, 2.0, 5.0, 10 ml or any range between about 0.001 and about 10 ml), which is much more suitable and desirable for PCR analysis. Only a small sample volume can be used in PCR analysis so concentration of the intact Norovirus in a small sample size will provide more accurate results.

After the neutralization buffer sample is contacted with the solid support comprising the ligand, the neutralization buffer sample is removed from the solid support using any suitable method, for example, separating magnetic beads with a magnet or draining or pipetting away the neutralization buffer sample from the solid support. The solid support can optionally be washed after the neutralization buffer sample is removed from the solid support.

Quantifying Norovirus RNA

Intact Norovirus virions remain in contact with the solid support. The RNA from these virions is detected and quantified. In general, one RNA copy number is equal to one virion. Methods for detecting and quantifying Norovirus RNA are known in the art and include, for example, polymerase chain reaction (PCR) amplification, RNA hybridization, gel electrophoresis, microarray or microchip analysis, in situ hybridization, and combinations thereof.

In one embodiment, the amount of Norovirus RNA can be determined by amplification of Norovirus RNA using, for example, quantitative reverse-transcription polymerase chain reaction (qRT-PCR). The levels of RNA transcripts can be quantified in comparison with an internal standard, for example, the level of RNA from a standard gene present in the same sample. The methods for qRT-PCR and variations thereof are within the skill in the art.

Any suitable primers can be used for the amplification of the Norovirus RNA. The primers are specific to Norovirus species and do not amplify other viral RNA outside of the genus Norovirus in the family Caliciviridae. It is within the skill in the art to generate primers specific to Norovirus. See, e.g., Wolf et al., Appl. Environ. Microbiol. 76:1388 (2010); Wolf et al., Appl. Environ. Microbiol. 73:5464 (2007). Primers can be of any suitable length, for example, between about 9 and about 70 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or any range of the values indicated herein) nucleotides.

The amount of Norovirus RNA in a sample also can be determined using Northern blotting, wherein total viral RNA is purified by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA then is immobilized on the filters by heating. Detection and quantification of specific RNA is accomplished using appropriately labeled DNA or RNA probes complementary to the RNA in question. See, e.g., Molecular Cloning: A Laboratory Manual, J. Sambrook et al., eds., 2nd edition, Cold Spring Harbor Laboratory Press, 1989, Chapter 7.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11. For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin, or an antibody), a fluorescent molecule, a chemiluminescent molecule, an enzyme, or the like.

Probes can be labeled to high specific activity by either the nick translation method of Rigby et al., J. Mol. Biol., 113: 237-251 (1977), or by the random priming method of Fienberg, Anal. Biochem., 132: 6-13 (1983). The latter can be a method for synthesizing ³²P-labeled probes of high specific activity from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization then can be performed by exposing hybridized filters to photographic film. Densitometric scanning of the photographic films exposed by the hybridized filters provides an accurate measurement of biomarker levels. Using another approach, RNA levels can be quantified by computerized imaging systems, such as the Molecular Dynamics 400-B 2D Phosphorimager (Amersham Biosciences, Piscataway, N.J., USA).

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA blotting hybridization techniques, determining the levels of RNA can be accomplished using the technique of in situ hybridization. The practice of the in situ hybridization technique is described in more detail in U.S. Pat. No. 5,427,916.

In some instances, it may be desirable to use microchip technology to detect Norovirus RNA. The microchip can be fabricated by techniques known in the art. For example, probe oligonucleotides of an appropriate length, e.g., 40 nucleotides, are 5′-amine modified at position C6 and printed using commercially available microarray systems, e.g., the GENEMACHINE® OMNIGRID® 100 Microarrayer and Amersham CODELINK® activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6 times SSPE/30% formamide at 25° C. for 18 hours, followed by washing in 0.75 times TNT at 37° C. for 40 minutes. At positions on the array, where the immobilized probe DNA recognizes a complementary target cDNA in the sample, hybridization occurs. The labeled target cDNA marks the exact position on the array where binding occurs, thereby allowing automatic detection and quantification. The output consists of a list of hybridization events, which indicate the relative abundance of specific cDNA sequences, and therefore the relative abundance of the corresponding complementary biomarker, in the subject sample. According to one embodiment, the labeled cDNA oligomer is a biotin-labeled cDNA prepared from a biotin-labeled primer. The microarray is then processed by direct detection of the biotin-containing transcripts and scanned utilizing conventional scanning methods. Image intensities of each spot on the array are proportional to the abundance of the corresponding RNA in the sample.

Antiviral activity of the test agent against Norovirus can be determined by comparing the amount of Norovirus RNA in the Norovirus positive sample contacted with the test agent with the amount of Norovirus RNA in a Norovirus positive sample that is not contacted with the test compound, a control, or reference data. If the amount of Norovirus RNA in the sample contacted with the test agent is less than the amount of Norovirus RNA in a control (e.g., Norovirus positive sample that is not contacted with the test compound, a positive Norovirus sample treated with a test agent known not to inactivate Norovirus, or reference data then test compound has antiviral activity against Norovirus). Reference data can be, for example, data indicating the amount of Norovirus expected to be present in a certain Norovirus positive sample. The amount of Norovirus RNA in the sample contacted with the test agent can be decreased by any suitable amount. For example, the amount of intact/infective Norovirus RNA in the sample contacted with the test compound can be reduced by at about least about 1 log (10 times) relative to the amount of Norovirus RNA in a control. The amount of Norovirus RNA in the sample contacted with the test agent can be reduced by, for example, at least about 2 log, 3 log, 4 log, 5 log, 6 log, 7 log, 8 log, 9 log, 10 log, or any range between about 1 log and about 10 log relative to the amount of intact/infective Norovirus RNA in a control. Antiviral activity is any reduction in the amount of Norovirus caused by a test agent. Additionally, other control samples, for example, samples without Norovirus or a test agent known to inactivate Norovirus can also be used in the methods of the invention.

In another embodiment of the invention, the methods of the invention can further comprise selecting a test agent that has at least about a 3, 4, 5, 6, 7, 8, 9, or 10 log reduction in the amount of Norovirus.

In another embodiment of the invention, a method of screening a test agent for antiviral activity against Norovirus is provided. The method comprises contacting a Norovirus-positive sample with a test agent to form a Norovirus-test agent sample. Neutralization buffer is added to the Norovirus-test agent sample to form a neutralization buffer sample. The neutralization buffer sample is contacted with one or more RNases either before addition of the neutralization buffer; after addition of the neutralization buffer; or before and after the addition of the neutralization buffer. Optionally, the one or more RNases can be added before or after the test agent is added. The amount of Norovirus in the sample is determined, wherein a decrease in the amount of Norovirus in comparison to a control is indicative of antiviral activity of the test compound.

### Kits

The invention also provides a kit for screening a test agent for antiviral activity against Norovirus. The kit comprises a solid support having a ligand that specifically binds Norovirus directly or indirectly bound to its surface(s), primers for amplification of Norovirus RNA, and a test or control sample comprising intact Norovirus. The kit also can comprise a positive control test agent that is known to inactivate Norovirus (e.g., sodium hypochlorite), and/or a negative control test agent that is known to not inactivate Norovirus (e.g., phosphate buffered saline-TWEEN®-20 (polysorbate)), or combinations thereof. The kit also can comprise directions describing the inventive screening method.

The invention also provides a kit for screening a test agent for antiviral activity against Norovirus. The kit comprises one or more RNases, primers for amplification of Norovirus RNA, and a test sample or control comprising intact Norovirus. The kit also can comprise a positive control test agent that is known to inactivate Norovirus (e.g., sodium hypochlorite), and/or a negative control test agent that is known to not inactivate Norovirus (e.g., phosphate buffered saline-TWEEN®-20 (polysorbate)), or combinations thereof. The kit also can comprise directions describing the inventive screening method.

### Surface Disinfection

In a further aspect, the invention is direct to a method of disinfecting a surface for Norovirus. The method includes selecting a test agent as a disinfectant for Norovirus by determining that the test agent has antiviral activity against Norovirus according to the methods described herein. When a test agent is found to have antiviral activity against Norovirus, the surface to be disinfected can be contacted with the test agent. In various aspects of the invention, the amount of Norovirus on the surface can be reduced by, for example, at least about 2 log, 3 log, 4 log, 5 log, 6 log, 7 log, 8 log, 9 log or 10 log. In one particular embodiment, the test agent is ACTICIDE™ disinfectant and the neutralization is accomplished by dilution with a suitable buffer, e.g., PBS, PBST.

The following examples further illustrate the invention but, of course, should not be construed as in any way limiting its scope.

### Example 1

This example demonstrates a method of screening test compounds for anti-Norovirus activity.

For this method, a Norovirus-positive human stool sample was allowed to dry on the surface of a material. A sterilized surface material (e.g., stainless steel, ceramic, porcelain, or fabric, etc.), in this case a round stainless steel plate, was placed into the center of a sterile deep Petri dish. 20 μl of Norovirus-positive human stool sample was added to the center of the stainless steel plate. The stool sample was dried onto the surface of the material for 30 minutes at room temperature.

100 μl of a test agent (e.g., a chemical disinfectant) was added on top of the dried stool sample. 100 μl of a neutral buffer, phosphate buffered saline-TWEEN® 20 (polysorbate) (PBST), was used as a negative control (in place of the test agent). 100 μl of 20% household bleach, (10500 ppm sodium hypochlorite, prepared fresh) was used as a positive control (in place of the test agent). The sample and the test agent, neutral buffer, and either 20% household bleach or 10500 ppm sodium hypochlorite were incubated at room temperature for as short as one minute to as long as 60 minutes.

40 ml of PBST was added over the surface to fully submerge the surface material and to dislodge the entire dried stool sample. 40 ml PBST acts as a neutralizer for the test compound by dilution. If necessary, the dried stool sample was scraped with the end of a sterile pipette tip. The mixture was shaken for 15 minutes (at ˜150 rpm) in an orbital shaker at room temperature.

The supernatant is transferred to a 50 ml screw cap centrifuge tube (or similar container). 10-100 μl of Norovirus specific antibody conjugated IMS beads were added, and the resulting mixture was incubated for 1-2 hours with end over rotation (5-10 RPM). The Norovirus specific antibody was monoclonal antibody P2B2 (Kim Laboratories, Inc., Rantoul, Ill.). A tube was placed in a magnetic stand for 15 minutes to capture the IMS beads. The supernatant was removed, with care being taken not to dislodge the IMS beads. During removal of supernatants, free RNA originating from stool, any PCR inhibitors, and RNA from disinfected, broken Norovirus are all removed and will interfere with downstream PCR analysis. The tube was removed from the magnetic stand and the beads were suspended in 140 μl of PBST. The RNA from live or intact Norovirus was isolated with Qiagen QIAamp Viral RNA kit according to the manufacturer's protocol. 5 μl of RNA can be used as template for qRT-PCR.

Results obtained using the above-described methods are set forth in Table 2.

TABLE 2
Hard Surface Testing (Porcelain Tile).

| Test Agent | Concentration | Log Reduction |
|---|---|---|
| Sodium Hypochlorite | 20% of household bleach | >5 |
| | by Clorox brand | |
| Silver nano-particles and | 1x working solution | 0.33 |
| citric acid solution | | |
| Silver nano-particles and | 10x working solution | 0.50 |
| citric acid solution | | |
| Silver nano-particles and | 100x working solution | 0.78 |
| citric acid solution | | |
| ACTICIDE ™ | 1x working solution | >5 |

Based on the inventive assay, sodium hypochlorite showed antiviral activity against Human Norovirus, while nanosilver solution (at any concentration tested) was ineffective against Norovirus. In addition, more than 2-3 weeks old 20% household bleach was not effective in Norovirus disinfection indicating that only freshly made bleach dilution is effective. ACTICIDE™, a proprietary disinfectant formula generated by Kim Laboratories, Rantoul, Ill., showed an efficacy of larger than 5 logs of viral reduction, proving that the compound contained within has significant anti-Norovirus activity.

### Example 2

This example demonstrates another method of screening test compounds for anti-Norovirus activity. For this method, Norovirus positive stool sample is in suspension.

20 μl of Norovirus positive human stool sample was added to a 50 ml conical bottom centrifuge tube. Immediately after the addition of the stool sample, 100 μl of the test agent was added. 100 μl of a neutral buffer, PBST, was used as a negative control and 100 μl of 20% household bleach or 10500 ppm sodium hypochlorite (prepared fresh) was used as a positive control. The sample and the test agent, neutral buffer, or 20% household bleach (10500 ppm sodium hypochlorite) were incubated at room temperature for as short as 1 min to as long as 60 min.

40 ml of PBST (a neutralization buffer) was added to each tube. 10-100 μl of Norovirus specific antibody conjugated immunomagnetic separation (IMS) beads were added, and the resulting mixture was incubated for 1-2 hours or overnight with end over rotation (5-10 RPM). The Norovirus specific antibodies were monoclonal antibodies P2B2 and/or P6G5 (Kim Laboratories, Inc., Rantoul, Ill.). A tube was placed in a magnetic stand for 15 minutes to capture the IMS beads. The supernatant was removed, with care being taken not to dislodge the IMS beads. During removal of supernatants, free RNA originating from stool, any PCR inhibitors, and RNA from disinfected, broken Norovirus were all removed and therefore did not interfere in downstream PCR analysis. The tube was removed from the magnetic stand and the beads were suspended in 140 μl of PBST. The RNA from intact Norovirus was isolated with Qiagen QIAamp Viral RNA kit according to the manufacturer's protocol. 5 μl of RNA can be used as template for qRT-PCR.

Results obtained using the above-described methods are set forth in Table 3.

TABLE 3
Suspension Testing.

| | | Log |
|---|---|---|
| Test Agent | Concentration | Reduction |
| Sodium Hypochlorite | 20% of household bleach | >5 |
| | by Clorox brand | |
| Silver nano-particles and | 100x working solution | 0.01 |
| Hydrogen Peroxide Solution | | |
| Silver nano-particles and | 10x working solution | 0.00 |
| Hydrogen Peroxide Solution | | |
| Silver nano-particles and | 1x working solution | 0.00 |
| Hydrogen Peroxide Solution | | |
| Silver nano-particles and citric | 1x working solution | 0.18 |
| acid solution | | |
| Silver nano-particles and citric | 10x working solution | 0.07 |
| acid solution | | |
| Silver nano-particles and citric | 100x working solution | 0.61 |
| acid solution | | |
| Organosilane quaternary | 1% | 1.04 |
| ammonium compound | | |
| Ethanol solution | 70% | 1.3 |
| Povidone Iodine | 5% | 1.1 |
| Citric Acid Solution | 0.6% | 1.26 |
| CLEAN & SHIELD ® surface | Hydrogen Peroxide | 1.25 |
| care product | | |
| ALOEUP ® Alcohol-Free Foam | Benzalkonium Chloride | 0 |
| Hand Sanitizer | (0.1%) | |
| MICROCIDE ™ TB spray | Quat (0.105%), Chloride | 0 |
| cleaner | Dioxide (0.2%) | |
| SNIPER ® biocide | Quat (0.085%) | 0 |
| CLOROX ® Wipes | Quat (0.145%) | 0 |
| GERMSTAR ® Noro hand | Ethyl alcohol (63%) | 1.1 |
| sanitizer | | |
| CLEANCIDE ® germicidal | Citric Acid (0.6%) | 1.54 |
| detergent | | |
| Zacsil | Isopropyl alcohol (58%), | 1.2 |
| | dimethyl AmCl | |
| ALPET D2 | Didecyl, dioctyl dimethyl | 0.13 |
| | AmCl (0.007) | |
| ACCEL TB ™ disinfectant | Hydrogen Peroxide | 1.06 |
| | (0.5%) | |
| VIRKON ® disinfectant | Potassium | 0.21 |
| | peroxymonosulfate | |
| | 20.4%, NaCl 1.5% | |
| Vital Oxide | Chlorine Dioxide 0.2%, | 0.71 |
| | Quat 0.125% | |
| STAPHACIDE ™ disinfectant | Silver 0.003%, Citric Acid | 0.3 |
| | 4.84% | |
| Sani-Spritz Spray | 4 types of Quat | 0 |
| LYSOL ® 4 in 1 disinfectant | 3.2% Lactic Acid | 1.8 |
| LYSOL ® can disinfectant | 0.1% Quat, EtOH | 0.32 |
| ACTICIDE ™ disinfectant | 1x working solution | >5 |

Based on the inventive assay, sodium hypochlorite showed antiviral activity against Norovirus in the suspension testing, while various chemical compounds including quaternary ammonium compounds, up to 70% alcohol, citric acid, lactic acid, potassium peroxymonosulfate, hydrogen peroxide, nanosilver solution (at any concentration tested) were demonstrated to be ineffective against Norovirus. In addition, more than 2-3 weeks old 20% household bleach was not effective in Norovirus disinfection indicating that only freshly made bleach dilution is effective (data not shown). ACTICIDE™, a proprietary disinfectant formula generated by Kim Laboratories, Rantoul, Ill., showed an efficacy of larger than 5 logs of viral reduction, proving that the compound contained within has significant anti-Norovirus activity. The inventive method facilitates testing of test compounds against Norovirus as opposed to a surrogate virus.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Certain embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method of screening a test agent for antiviral activity against Norovirus comprising
(a) contacting a Norovirus-positive sample with a test agent to form a Norovirus-test agent sample;
(b) adding neutralization buffer to the Norovirus-test agent sample to form a neutralization buffer sample;
(c) contacting the neutralization buffer sample with a solid support,
(d) separating the solid support from the neutralization buffer sample; and
(e) determining the amount of Norovirus associated with the solid support, wherein a decrease in the amount of Norovirus in comparison to a control is indicative of antiviral activity of the test compound.

2. The method of claim 1, further comprising selecting a test agent that has at least a 1, 2, or 3 log reduction in the amount of Norovirus.

3. The method of claim 1, further comprising contacting the neutralization buffer sample with one or more RNases (i) before the neutralization buffer is added to neutralization buffer sample; (ii) after the neutralization buffer is added to the neutralization buffer sample; or (iii) before and after the neutralization buffer is added to the neutralization buffer sample.

4. The method of claim 1, wherein the solid support comprises a material that non-specifically binds viruses.

5. The method of claim 4, wherein the material that non-specifically binds viruses is polystyrene or polypropylene or a combination thereof.

6. The method of claim 1, wherein the solid support comprises a ligand specific for Norovirus.

7. The method of claim 6, wherein the ligand specific for Norovirus is a monoclonal antibody, polyclonal antibody, cell receptor, or a histo-blood group antigen (HBGA).

8. The method of claim 6, wherein the solid support comprises immunomagnetic separation (IMS) beads, magnetic beads, hydrophobic beads, nitrocellulose, a filter, a plastic sheet or slide, glass beads, controlled pore silicate glass beads, a tube, a microtiter plate, a glass slide, a microchip array, a well or depression, ceramic, metal, resin, gel silicon, microspheres, or a combination thereof.

9. The method of claim 1, wherein the amount of Norovirus in the sample is determined using microarray analysis, PCR amplification, RNA hybridization, gel electrophoresis, quantitative reverse transcription PCR (qRT-PCR), or combinations thereof.

10. The method of claim 1, wherein the Norovirus positive sample is stool, vomitus, tissue, sputum, blood, plasma, serum, food, water, an environmental sample, a laboratory sample or any combinations thereof.

11. The method of claim 1, wherein the Norovirus positive sample is from a human.

12. A kit for screening for a compound for antiviral activity against Norovirus comprising a solid support having a ligand that specifically binds Norovirus directly or indirectly bound to its surface, primers for amplification of Norovirus RNA, and a sample comprising active, infectious Norovirus.

13. The kit of claim 12, further comprising one or more RNases.

14. A method of screening a test agent for antiviral activity against Norovirus comprising:
(a) contacting a Norovirus-positive sample with a test agent to form a Norovirus-test agent sample;
(b) adding neutralization buffer to the Norovirus-test agent sample to form a neutralization buffer sample;
(c) contacting the neutralization buffer sample with one or more RNases either (i) before the neutralization buffer is added to the neutralization buffer sample; (ii) after the neutralization buffer is added to the neutralization buffer sample; or (iii) before and after the neutralization buffer is added to the neutralization buffer sample; and
(e) determining the amount of Norovirus in the neutralization buffer sample; and
(f) determining that the test agent has antiviral activity when the amount of Norovirus in the neutralization buffer sample is less than the amount of Norovirus in a control sample.

15. The method of claim 14, further comprising selecting a test agent that has at least a 1, 2, or 3 or more log reduction in the amount of Norovirus.

16. The method of claim 14, wherein the amount of Norovirus in the sample is determined using microarray analysis, PCR amplification, quantitative reverse transcription PCR (qRT-PCR), RNA hybridization, gel electrophoresis, or combinations thereof.

17. The method of claim 14, wherein the Norovirus positive sample is stool, vomitus, tissue, sputum, blood, plasma, serum, food, water, an environmental sample, a laboratory sample or any combinations thereof.

18. The method of claim 14, wherein the Norovirus positive sample is from a human.

19. A method of disinfecting a surface against Norovirus comprising:
(a) selecting a test agent as a disinfectant for Norovirus by determining that the compound has antiviral activity against Norovirus according to the method of claim 1,
(b) contacting the surface with the test agent to disinfect the surface against Norovirus.

20. The method of claim 19, wherein the test agent is ACTICIDE™ disinfectant.
